(19) United States
(12) Patent Application Publication
(10) Pub. No.: US 2024/0182646 A1
(43) Pub. Date: Jun. 6, 2024

(54) BISMALEIMIDE COMPOUND, RESIN VARNISH, AND PRODUCTION METHOD THEREOF

(71) Applicant: Shin-Etsu Chemical Co., Ltd., Tokyo (JP)

(72) Inventors: Yuki KUDO, Annaka-shi (JP); Yoshihiro TSUTSUMI, Annaka-shi (JP); Hiroyuki IGUCHI, Annaka-shi (JP); Atsushi TSUURA, Annaka-shi (JP)

(73) Assignee: Shin-Etsu Chemical Co., Ltd., Tokyo (JP)

(21) Appl. No.: 18/515,682

(22) Filed: Nov. 21, 2023

(30) Foreign Application Priority Data
Nov. 22, 2022 (JP) 2022-186292

(51) Int. Cl. C08G 73/10; C09D 7/20; C09D 179/08

(52) U.S. Cl. CPC C08G 73/1071; C08G 73/1021; C08G 73/1032; C09D 7/20; C09D 179/08

(57) ABSTRACT

Provided is a bismaleimide compound where the compound itself has a superior solubility in various solvents; a resin composition containing this compound has a superior flexibility; and a cured product of such composition has a low relative permittivity and dielectric tangent, a low thermal expansion coefficient, and a superior heat resistance due to a high glass-transition temperature. The bismaleimide compound is represented by the following formula (1):

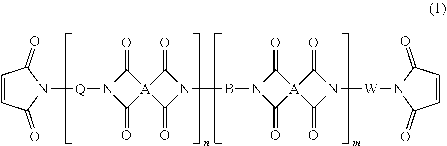

wherein A independently represents a cyclic structure-containing tetravalent organic group; B independently represents a dimer acid frame-derived divalent hydrocarbon group; Q independently represents an aromatic ring-containing divalent group that has a fluorene frame or indene frame; W is B or Q; n is 1 to 100; m is 1 to 100; no restrictions are imposed on an order of each repeating unit identified by n and m, and a bonding pattern thereof may be alternate, block or random.

### BACKGROUND OF THE INVENTION

### Field of the Invention

The present invention relates to a bismaleimide compound, a resin varnish, and a production method thereof.

### Background Art

In recent years, a next-generation communication system known as 5G (millimeterwave region of 26 to 80 GHz) has prevailed, and even a next-next-generation communication system called 6G is already under development, where attempts are being made to realize a communication with a higher speed, a larger capacity and a lower latency than ever before. The realization of these communication systems requires materials for use in a high-frequency band of 3 to 80 GHz, and it is critical to reduce a transmission loss as a countermeasure for noise.

A transmission loss is a sum of a conductor loss and a dielectric loss; it is necessary to lower the roughness of the surface of a metal foil used to reduce a conductor loss. Meanwhile, since a dielectric loss is proportionate to a product of a square root of a relative permittivity and a dielectric tangent, as an insulating material, it is demanded that there be developed one with excellent dielectric properties (i.e. with a low relative permittivity and a low dielectric tangent).

Further, in the cases of printed wiring boards and electronic parts in which insulating materials are used, there is a demand for a material having a high heat resistance, i.e., a high glass-transition temperature (Tg) because a reflow step is required at the time of mounting.

Particularly, there is a demand for an insulating material that has excellent dielectric properties and is for use in substrates. In the cases of rigid substrates, reactive polyphenylene ether resins (PPE) are becoming used; and in the cases of flexible printed-circuit boards (FPC), liquid crystal polymers (LCP) and products called modified polyimides (MPI) with improved properties are becoming used.

While these materials have a number of superior characteristics, it is also true that they have many problems. For example, in the case of a reactive PPE resin, while it has superior dielectric properties and a high Tg, there will be obtained a brittle cured product and thus incurred a poor handling property, which limits the resin's use to prepregs or the like (e.g., WO2019/65940). In the case of LCP, there have been disclosed many inventions such as those with higher performances, and base films or coverlay films for use in FPCs employing LCPs (e.g., JP-A-2013-74129); however, LCP still has many flaws that need to be improved, such as the fact that its use is limited due to a difficulty in mass production matching up with demand, and the fact that an adhesive agent superior in dielectric properties is required in order to be bonded to a copper-clad laminate. MPI has a problem in processibility as a high temperature of 300° C. or higher is needed for imidization of polyamic acid (polyamide acid) (e.g., JP-A-2019-104818).

In this regard, maleimide resin has gathered attention in recent years as a material having both heat resistance and dielectric properties. Even among maleimide resins, bismaleimide resins are common. Many bismaleimide resins are known to have low molecular weights; while bismaleimide resins are superior in heat resistance due to a high Tg, for example, they have a low solvent solubility and will provide a hard and brittle cured product. Further, since the dielectric properties of a bismaleimide resin are also insufficient as compared to LCP and MPI, there has been a strong demand for developing a resin that is superior in dielectric properties and solvent solubility and has a film property while maintaining a heat resistance.

With regard to such demand, reports have been made on uses of dimer diamine frame (dimer acid-derived frame)-containing maleimide compounds (particular types of maleimide compounds) as main resins for use in substrates (JP-A-2016-131243, JP-A-2016-131244, WO2016/114287, JP-A-2018-201024, and JP-A-2021-25051). Unlike the properties of a general maleimide resin, a maleimide compound of a particular type is significantly superior in dielectric properties, has a property of being flexible, and is superior in adhesiveness to an adherend such as metal. However, since these maleimide compounds have a low Tg and a high thermal expansion coefficient (CTE), changes in dielectric properties are significant and heat resistance is not sufficient under a high-temperature environment.

### SUMMARY OF THE INVENTION

Thus, it is an object of the present invention to provide a bismaleimide compound where the compound itself has a superior solubility in various solvents; a resin composition containing this compound has a superior flexibility; and a cured product of such composition has a low relative permittivity and dielectric tangent, a low thermal expansion coefficient, and a superior heat resistance due to a high glass-transition temperature.

The inventors of the present invention diligently conducted a series of studies to solve the above problems, and completed the invention by finding that the bismaleimide compound shown below was able to achieve the above object.

That is, the present invention relates to the following <1> to <9>.

<1>

A bismaleimide compound represented by the following formula (1):

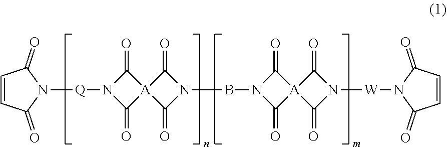

wherein A independently represents a cyclic structure-containing tetravalent organic group; B independently represents a dimer acid frame-derived divalent hydrocarbon group; Q independently represents an aromatic ring-containing divalent group; W is B or Q; n is 1 to 100; m is 1 to 100; no restrictions are imposed on an order of each repeating unit identified by n and m, and a bonding pattern thereof may be alternate, block or random, and

wherein Q has a fluorene frame or indene frame expressed by any of the following formula (2-1) or (2-2):

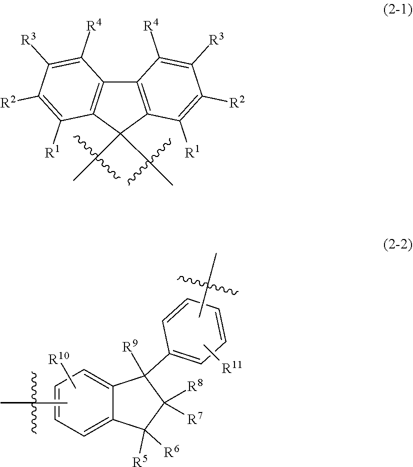

wherein in the formula (2-1), each of R¹, R², R³ and R⁴ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group, and

wherein in the formula (2-2), each of R⁵, R⁶, R⁷, R⁸, R⁹, R¹⁰ and R¹¹ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group.

<2>

The bismaleimide compound according to <1>, wherein A in the formula (1) is a tetravalent organic group represented by the following structural formula (5):

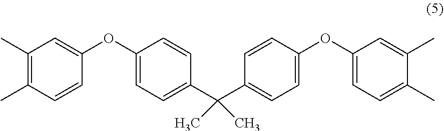

wherein bonds that are yet unbonded to substituent groups are to be bonded to carbonyl carbons forming cyclic imide structures in the formula (1).

<3>

The bismaleimide compound according to <1> or <2>, wherein the bismaleimide compound represented by the formula (1) has a number average molecular weight of 3,000 to 50,000.

<4>

The bismaleimide compound according to any one of <1> to <3>, wherein in the bismaleimide compound represented by the formula (1), the bonding pattern of each repeating unit identified by n and m is block.

<5>

A resin varnish containing the bismaleimide compound according to any one of <1> to <4>

and a solvent.

<6>

The resin varnish according to <5>, wherein the solvent is an aromatic solvent.

The resin varnish according to <6>, wherein the aromatic solvent is toluene, xylene, or anisole.

<8>

A method for producing the bismaleimide compound according to any one of <1> to <4>, comprising:
a step A of synthesizing an amic acid with an acid anhydride represented by the following formula (6) and an aromatic ring-containing diamine represented by the following formula (7), and then performing cyclodehydration;
a step B subsequent to the step A, which is a step of synthesizing an amic acid with a reactant obtained in the step A and a dimer acid frame-derived diamine represented by the following formula (8), and then performing cyclodehydration; and
a step C subsequent to the step B, which is a step of synthesizing a maleamic acid with a reactant obtained in the step B and a maleic anhydride, and then performing cyclodehydration to block molecular chain ends,

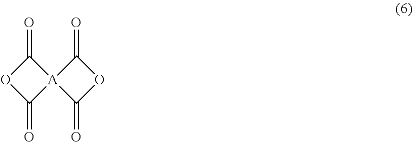

wherein A represents a cyclic structure-containing tetravalent organic group;

H₂N-Q-NH₂ (7)

wherein Q represents an aromatic ring-containing divalent group that has a fluorene frame or indene frame expressed by any of the following formula (2-1) or (2-2),

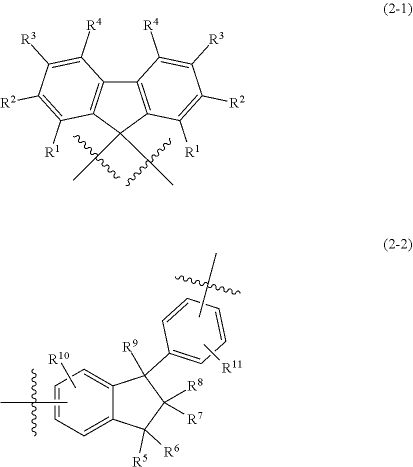

wherein in the formula (2-1), each of R¹, R², R³ and R⁴ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group, and

wherein in the formula (2-2), each of R⁵, R⁶, R⁷, R⁸, R⁹, R¹⁰ and R¹¹ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group;

H₂N—B—NH₂ (8)

wherein B represents a dimer acid frame-derived divalent hydrocarbon group.

<9>

A method for producing the bismaleimide compound according to any one of <1> to <4>, comprising:
a step A′ of synthesizing an amic acid with an acid anhydride represented by the following formula (6) and a diamine represented by the following formula (8), and then performing cyclodehydration;
a step B′ subsequent to the step A′, which is a step of synthesizing an amic acid with a reactant obtained in the step A′ and an aromatic ring-containing diamine represented by the following formula (7), and then performing cyclodehydration; and
a step C′ subsequent to the step B′, which is a step of synthesizing a maleamic acid with a reactant obtained in the step B′ and a maleic anhydride, and then performing cyclodehydration to block molecular chain ends,

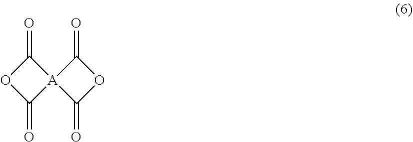

wherein A represents a cyclic structure-containing tetravalent organic group;

H₂N-Q-NH₂ (7)

wherein Q represents an aromatic ring-containing divalent group that has a fluorene frame or indene frame expressed by any of the following formula (2-1) or (2-2),

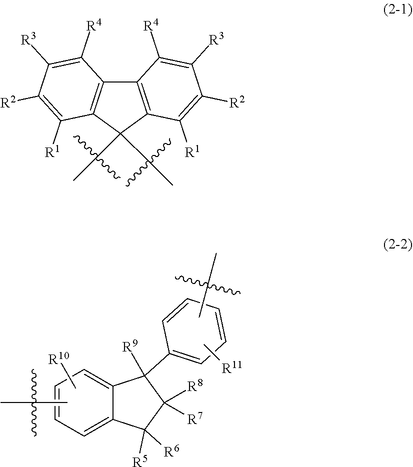

wherein in the formula (2-1), each of R¹, R², R³ and R⁴ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group, and

wherein in the formula (2-2), each of R⁵, R⁶, R⁷, R⁸, R⁹, R¹⁰ and R¹¹ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group;

H₂N—B—NH₂ (8)

wherein B represents a dimer acid frame-derived divalent hydrocarbon group.

The bismaleimide compound of the present invention has an excellent solubility in various kinds of solvents. Further, a cured film produced from a resin composition containing the bismaleimide compound of the present invention has an excellent flexibility. Furthermore, a resin composition containing the bismaleimide compound of the present invention can be turned into a cured product exhibiting excellent dielectric properties, i.e., a low relative permittivity and dielectric tangent at high frequencies, a high glass-transition temperature, a low thermal expansion coefficient, and an excellent heat resistance and moisture resistance.

As an insulating material intended for high-frequency band uses, the bismaleimide compound of the present invention can be favorably used in, for example, printed-wiring boards and electronic parts.

### BRIEF DESCRIPTION OF THE DRAWINGS

FIG. 1 is a ¹H-NMR spectrum of a bismaleimide compound synthesized in a working example 1.

FIG. 2 is an IR spectrum of the bismaleimide compound synthesized in the working example 1.

FIG. 3 is a ¹H-NMR spectrum of a bismaleimide compound synthesized in a working example 2.

FIG. 4 is an IR spectrum of the bismaleimide compound synthesized in the working example 2.

FIG. 5 is a ¹H-NMR spectrum of a bismaleimide compound synthesized in a working example 3.

FIG. 6 is an IR spectrum of the bismaleimide compound synthesized in the working example 3.

FIG. 7 is a ¹H-NMR spectrum of a bismaleimide compound synthesized in a working example 4.

FIG. 8 is an IR spectrum of the bismaleimide compound synthesized in the working example 4.

FIG. 9 is a ¹H-NMR spectrum of a bismaleimide compound synthesized in a working example 5.

FIG. 10 is an IR spectrum of the bismaleimide compound synthesized in the working example 5.

### DETAILED DESCRIPTION OF THE INVENTION

The present invention is described in detail hereunder.

A bismaleimide compound of the present invention is represented by the following formula (1).

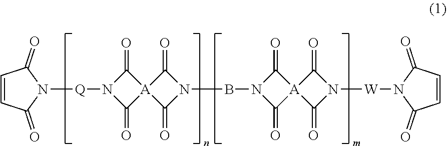

In the formula (1), A independently represents a cyclic structure-containing tetravalent organic group. B independently represents a dimer acid frame-derived divalent hydrocarbon group. Q independently represents an aromatic ring-containing divalent group that has a fluorene frame or indene frame expressed by any of the following formula (2-1) or (2-2). W is B or Q. n is 1 to 100, m is 1 to 100. Further, no restrictions are imposed on the order of each repeating unit identified by n and m, and the bonding pattern thereof may be alternate, block or random.

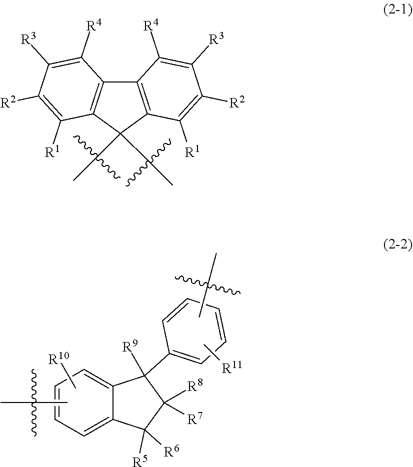

In the formula (2-1), each of R¹, R², R³ and R⁴ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group.

As the alkyl group having 1 to 5 carbon atoms that is represented by R¹, R², R³ and R⁴ in the formula (2-1), there may be listed, for example, a methyl group, an ethyl group, a n-propyl group, an isopropyl group, a n-butyl group, a sec-butyl group, an isobutyl group, a tert-butyl group, and a pentyl group.

As the (hetero)aryl group having 4 to 10 carbon atoms that is represented by R¹, R², R³ and R⁴ in the formula (2-1), there may be listed, for example, an aryl group having 6 to 10 carbon atoms, such as a phenyl group, a tolyl group, a xylyl group, and a naphthyl group; and a hetero aryl group having 4 to 10 carbon atoms, such as a furyl group, a thienyl group, a pyridyl group, and an indolyl group.

As the alkoxy group that is represented by R¹, R², R³ and R⁴ in the formula (2-1), there may be listed, for example, a methoxy group, an ethoxy group, a n-propyloxy group, an isopropyloxy group, a n-butyloxy group, an isobutyloxy group, a t-butyloxy group, a n-pentyloxy group, an isopentyloxy group, a hexyloxy group, a benzyloxy group, a phenethyloxy group, an allyloxy group, a phenyloxy group, a tolyloxy group, a xylyloxy group, a naphthyloxy group, a furyloxy group, a thienyloxy group, a pyridyloxy group, and an indolyloxy group.

As the halogeno group that is represented by R¹, R², R³ and R⁴ in the formula (2-1), there may be listed, for example, a fluoro group, a chloro group, a bromo group, and an iodo group.

As R¹, R², R³ and R⁴ in the formula (2-1), preferred is a hydrogen atom; specific examples of Q that has the fluorene frame expressed by the formula (2-1) may include those represented by the following structural formulae.

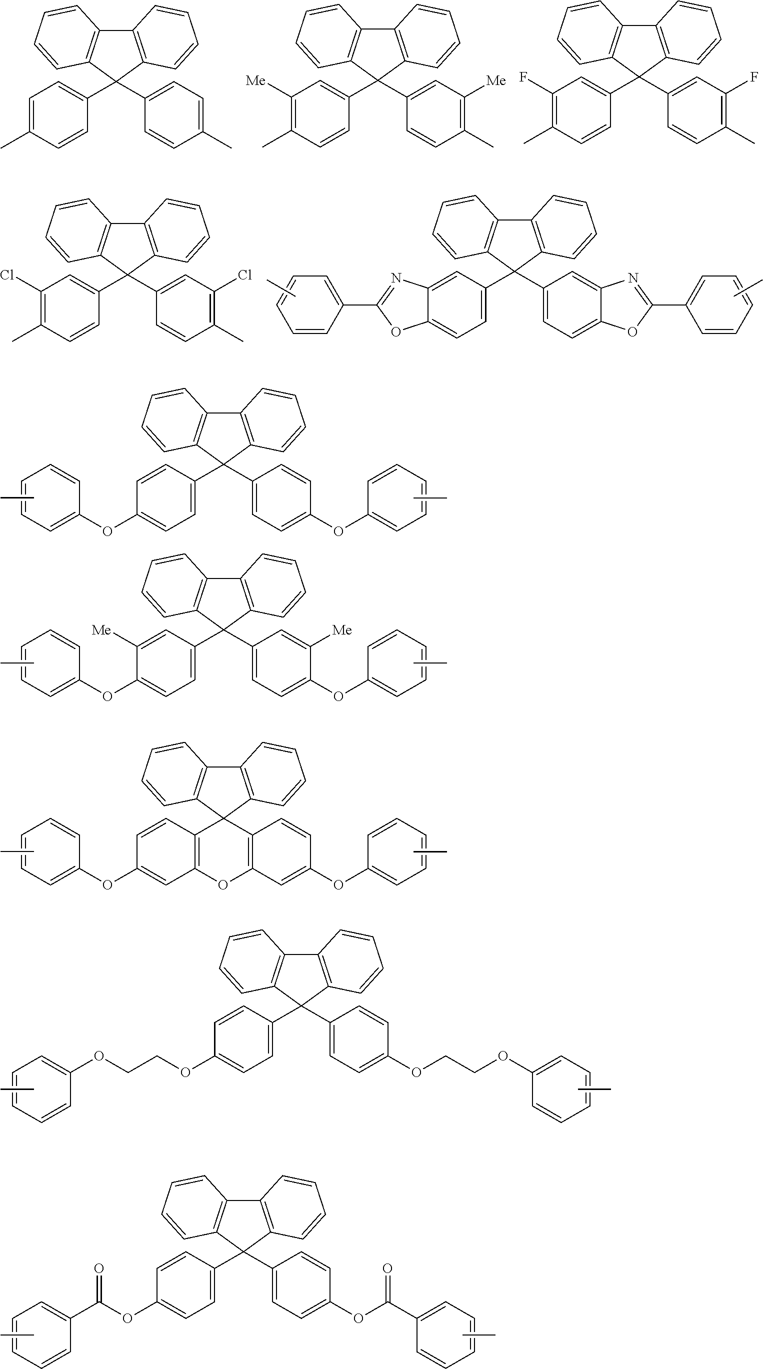

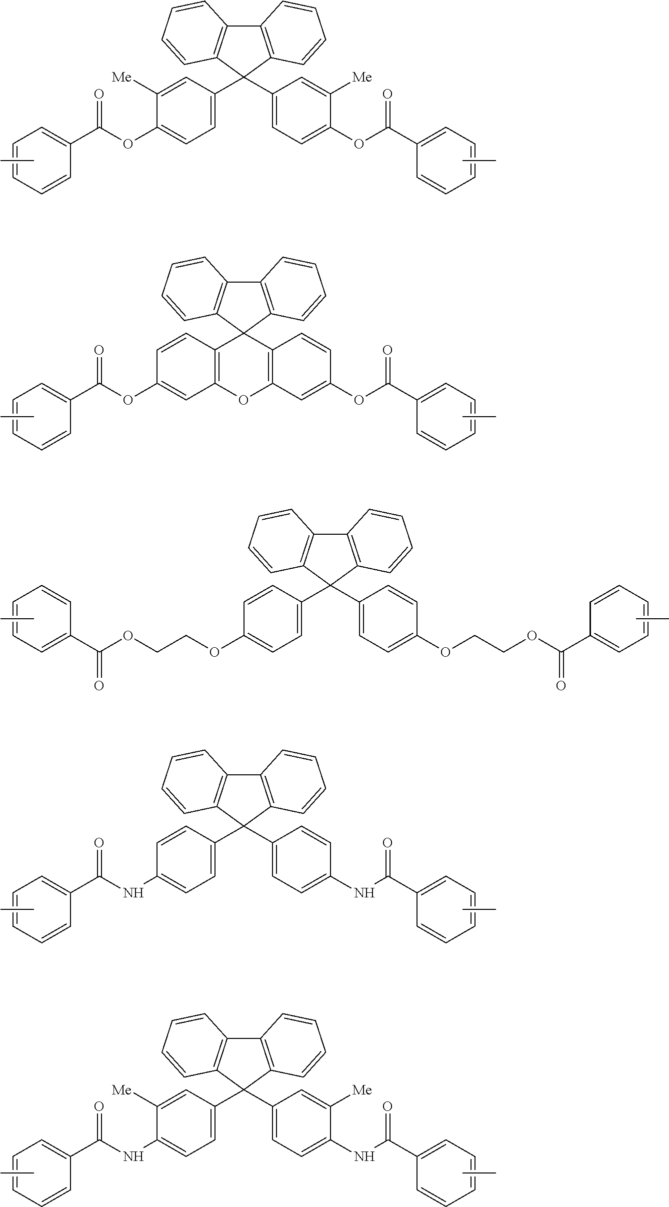

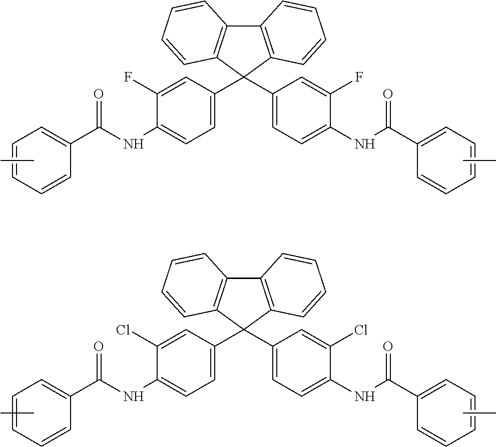

In the above structural formulae, bonds that are yet unbonded to substituent groups are to be bonded to nitrogen atoms forming cyclic imide structures in the formula (1).

As Q that has the fluorene frame expressed by the formula (2-1), groups represented by the following structural formulae are particularly preferred.

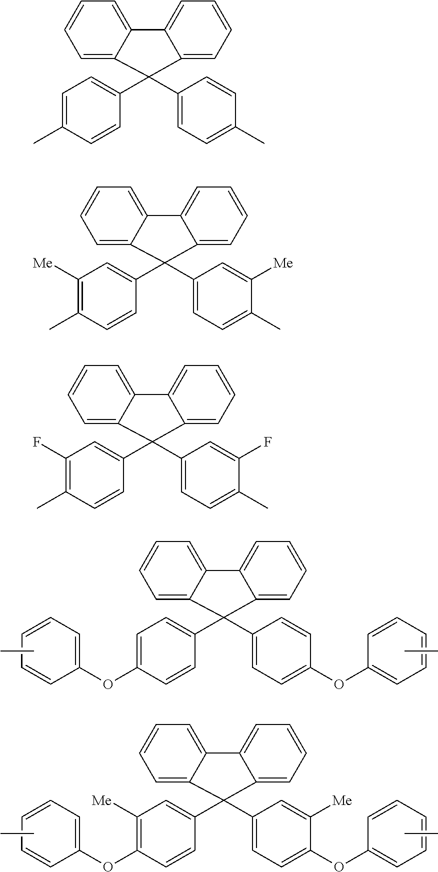

In the formula (2-2), each of R⁵, R⁶, R⁷, R⁸, R⁹, R¹⁰ and R¹¹ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group.

As the alkyl group having 1 to 5 carbon atoms that is represented by R⁵, R⁶, R⁷, R⁸, R⁹, R¹⁰ and R¹¹ in the formula (2-2), there may be listed, for example, a methyl group, an ethyl group, a n-propyl group, an isopropyl group, a n-butyl group, a sec-butyl group, an isobutyl group, a tert-butyl group, and a pentyl group.

As the (hetero)aryl group having 4 to 10 carbon atoms that is represented by R⁵, R⁶, R⁷, R⁸, R⁹, R¹⁰ and R¹¹ in the formula (2-2), there may be listed, for example, an aryl group having 6 to 10 carbon atoms, such as a phenyl group, a tolyl group, a xylyl group, and a naphthyl group; and a hetero aryl group having 4 to 10 carbon atoms, such as a furyl group, a thienyl group, a pyridyl group, and an indolyl group.

As the alkoxy group that is represented by R⁵, R⁶, R⁷, R⁸, R⁹, R¹⁰ and R¹¹ in the formula (2-2), there may be listed, for example, a methoxy group, an ethoxy group, a n-propyloxy group, an isopropyloxy group, a n-butyloxy group, an isobutyloxy group, a t-butyloxy group, a n-pentyloxy group, an isopentyloxy group, a hexyloxy group, a benzyloxy group, a phenethyloxy group, an allyloxy group, a phenyloxy group, a tolyloxy group, a xylyloxy group, a naphthyloxy group, a furyloxy group, a thienyloxy group, a pyridyloxy group, and an indolyloxy group.

As the halogeno group that is represented by R⁵, R⁶, R⁷, R⁸, R⁹, R¹⁰ and R¹¹ in the formula (2-2), there may be listed, for example, a fluoro group, a chloro group, a bromo group, and an iodine group.

As R⁵, R⁶, R⁷, R⁸, R⁹, R¹⁰ and R¹¹ in the formula (2-2), preferred are a hydrogen atom and an alkyl group having 1 to 5 carbon atoms, particularly preferred are a hydrogen atom and a methyl group. Further, specific examples of Q that has the indene frame expressed by the formula (2-2) may include those represented by the following structural formulae.

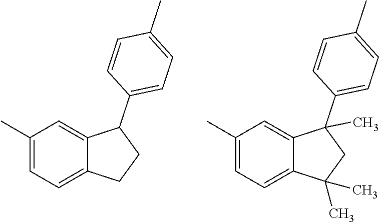

In the above structural formulae, bonds that are yet unbonded to substituent groups are to be bonded to nitrogen atoms forming cyclic imide structures in the formula (1).

The aromatic ring-containing divalent group represented by Q is a group derived from an aromatic ring-containing diamine or the like in a later-described production method. Examples of such aromatic ring-containing diamine include 9,9-bis(4-aminophenyl)fluorene (hereinafter also referred to as FDA), 9,9-bis(4-amino-3-methylphenyl)fluorene, 9,9-bis(4-amino-3-fluorophenyl)fluorene (hereinafter also referred to as FFDA), 9,9-bis(4-amino-3-chlorophenyl)fluorene, 9,9-bis(4-amino-3-hydroxyphenyl)fluorene, 9,9-bis[4-(4-aminophenoxy)phenyl]fluorene, 1-(4-aminophenyl)-2,3-dihydro-1,3,3-trimethyl-1H-indene-5-amine (hereinafter also referred to as PIDA), and 3-(4-aminophenyl)-2,3-dihydro-3-methyl-1,1-diphenyl-1H-indene-5-amine 2-(4-aminophenyl)-1-ethyl-2,3-dihydro-3-methyl-1H-indene-5-amine 1,3,3-tris(4-aminophenyl)-2,3-dihydro-1-methyl-1H-indene-5-amine. Depending on, for example, a purpose or intended use, any one kind of these diamines may be used alone, or two or more kinds of them may be used in combination. In order for a resin composition containing the bismaleimide compound of the formula (1) to exhibit superior dielectric properties, a high glass-transition temperature, and a low thermal expansion coefficient, it is preferred that the aromatic ring-containing diamine be FDA, FFDA, or PIDA.

In the formula (1), A independently represents a cyclic structure-containing tetravalent organic group, and is a group derived from a tetracarboxylic dianhydride monomer. Examples of such tetracarboxylic dianhydride monomer include pyromellitic dianhydride, 4,4′-carbonyldiphthalic dianhydride, 4,4′-oxydiphthalic dianhydride, 3,4′-oxydiphthalic anhydride, 4,4′-biphthalic dianhydride, 4,4′-(4,4′-isopropylidenediphenoxy)diphthalic dianhydride, 1,2,3,4-cyclobutanetetracarboxylic dianhydride, 1,2,4,5-cyclohexanetetracarboxylic dianhydride, 1,2,3,4-cyclopentanetetracarboxylic dianhydride, 9,9-bis(3,4-dicarboxyphenyl)fluorene dianhydride, 4,4′-(hexafluoroisopropylidene)diphthalic anhydride, dicyclohexyl-3,4,3′,4′-tetracarboxylic dianhydride, norbornane-2-spiro-α-cyclopentanone-′′-spiro-2″-norbornane-5,5″,6,6″-tetracarboxylic dianhydride, 3,3′,4,4′-benzophenonetetracarboxylic dianhydride, 4,4′-(ethyne-1,2-diyl)diphthalic dianhydride, 3-(carboxymethyl)-1,2,4-cyclopentanetricarboxylic acid 1,4:2,3-dianhydride, 2,3,6,7-naphthalenetetracarboxylic acid 2,3:6,7-dianhydride, naphthalene-1,4,5,8-tetracarboxylic dianhydride, bis(1,3-dioxo-1,3-dihydroisobenzofuran-5-carboxylic acid) 1,4-phenylene, and 3,4,9,10-perylenetetracarboxylic dianhydride. Depending on, for example, a purpose or intended use, any one kind of these acid anhydrides may be used alone, or two or more kinds of them may be used in combination. In order for the resin composition containing the bismaleimide compound of the formula (1) to exhibit superior dielectric properties and a superior solubility in a solvent, preferred is 4,4′-(4,4′-isopropylidenediphenoxy)diphthalic dianhydride.

In the formula (1), B independently represents one or more kinds of dimer acid frame-derived divalent hydrocarbon group. A dimer acid here refers to a liquid dibasic acid whose main component is a dicarboxylic acid having 36 carbon atoms, which is produced by dimerizing an unsaturated fatty acid having 18 carbon atoms and employing a natural substance such as a vegetable fat or oil as its raw material; a dimer acid frame may contain multiple structures as opposed to one single type of frame, and there exist several types of isomers. Typical dimer acids are categorized under the names of (a) linear type, (b) monocyclic type, (c) aromatic ring type, and (d) polycyclic type.

In this specification, a dimer acid frame refers to a group induced from a dimer diamine having a structure established by substituting the carboxy groups in such dimer acid with primary aminomethyl groups.

That is, the dimer acid frame-derived divalent hydrocarbon group represented by B in the formula (1) may for example be, but is not limited to a branched divalent hydrocarbon group obtained by substituting the two carboxy groups in any of the following dimer acids (a) to (d) with methylene groups.

Further, as for such dimer acid frame-derived hydrocarbon group, more preferred from the perspectives of heat resistance and reliability of a cured product are those having a structure with a reduced number of carbon-carbon double bonds in the dimer acid frame-derived hydrocarbon group due to a hydrogenation reaction.

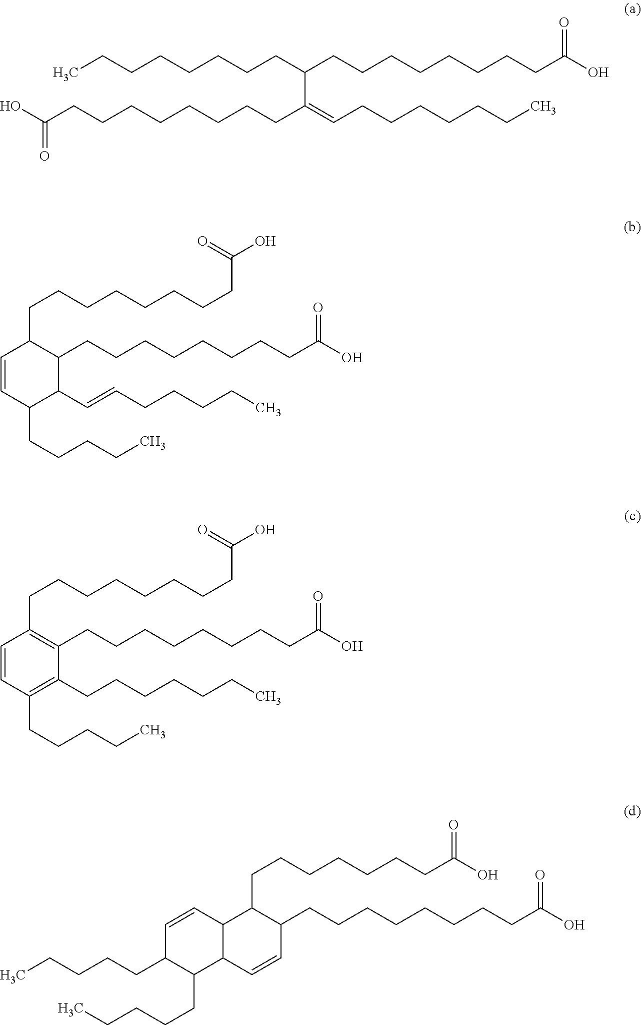 [SMILES: CCCCCCC/C=C(/CCCCCCCCC(=O)O)C(CCCCCCCC)CCCCCCCCC(=O)O ; CCCCC/C=C/C1C(CCCCC)C=CC(CCCCCCCCC(=O)O)C1CCCCCCCCC(=O)O ; CCCCCCCc1c(CCCCC)ccc(CCCCCCCCC(=O)O)c1CCCCCCCCC(=O)O ; CCCCCC1C=CC2C(C=CC(CCCCCCCCC(=O)O)C2CCCCCCCC(=O)O)C1CCCCC]

As mentioned above, a dimer acid frame may contain multiple structures; in this specification, the dimer acid frame-derived divalent hydrocarbon group may be expressed as —C₃₆H₇₀— as its average structure.

In the formula (1), W is B or Q. Whether W will be B or Q is determined by a difference(s) in a later-described production method.

In the formula (1), n is 1 to 100, preferably 1 to 50, more preferably 1 to 10. Further, m is 1 to 100, preferably 1 to 50, more preferably 1 to 10.

There are no particular restrictions on a number average molecular weight (Mn) of the bismaleimide compound of the present invention; it is preferred that the bismaleimide compound of the present invention have a number average molecular weight of 3,000 to 50,000, more preferably 3,500 to 20,000, even more preferably 4,000 to 10,000. If the number average molecular weight thereof is within these ranges, a resin composition containing the bismaleimide compound of the present invention will not exhibit an excessively high viscosity, and a cured product of such resin composition shall exhibit a high hardness.

Here, the term “number average molecular weight (Mn)” referred to in this specification means a number average molecular weight measured by GPC under the following conditions, using polystyrene as a reference substance.

### [GPC Measurement Condition]

Developing solvent: tetrahydrofuran
Flow rate: 0.6 mL/min
Column: TSK Guardcolumn Super H-L
TSKgel Super H4000 (6.0 mmI.D.×15 cm×1)
TSKgel Super H3000 (6.0 mmI.D.×15 cm×1)
TSKgel Super H2000 (6.0 mmI.D.×15 cm×2) (all by TOSOH CORPORATION)
Column temperature: 40° ° C.
Sample injection volume: 20 μL (Sample concentration: 0.5% by mass-tetrahydrofuran solution)
Detector: differential refractometer (RI)

There are no restrictions on an order in which units identified by n and m are repeated; a bonding pattern of each of these units n and m may be alternate, block or random. Here, block is preferred.

### Method for Producing Bismaleimide Compound

There are no particular restrictions on a method for producing the bismaleimide compound of the present invention. The compound may, for example, be efficiently produced by any one of the two methods shown below.

### Production Method 1

A first method for producing the bismaleimide compound includes a step A of synthesizing an amic acid with an acid anhydride and an aromatic ring-containing diamine, and then performing cyclodehydration; a step B subsequent to the step A, which is a step of synthesizing an amic acid with the reactant obtained in the step A and a dimer acid frame-derived diamine, and then performing cyclodehydration; and a step C subsequent to the step B, which is a step of synthesizing a maleamic acid with the reactant obtained in the step B and a maleic anhydride, and then performing cyclodehydration to block molecular chain ends with maleimide groups.

The acid anhydride used in the step A is represented by the following formula (6):

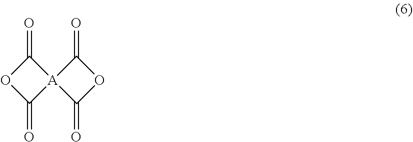

wherein A is defined as above in the formula (1).

The aromatic ring-containing diamine used in the step A is represented by the following formula (7):

H₂N-Q-NH₂ (7)

wherein Q is defined as above in the formula (1).

The dimer acid frame-derived diamine used in the step B is represented by the following formula (8):

H₂N—B—NH₂ (8)

wherein B is defined as above in the formula (1).

### Production Method 2

A second method for producing the bismaleimide compound includes a step A′ of synthesizing an amic acid with the acid anhydride represented by the formula (6) and the dimer acid frame-derived diamine represented by the formula (8), and then performing cyclodehydration; a step B′ subsequent to the step A′, which is a step of synthesizing an amic acid with the reactant obtained in the step A′ and the aromatic ring-containing diamine represented by the formula (7), and then performing cyclodehydration; and a step C′ subsequent to the step B′, which is a step of synthesizing a maleamic acid with the reactant obtained in the step B′ and a maleic anhydride, and then performing cyclodehydration to block molecular chain ends with maleimide groups.

The two production methods have now been described. As a basic pattern, the bismaleimide compound can be obtained by the step A (or step A′) of synthesizing an amic acid with a tetracarboxylic dianhydride and a diamine, and then performing cyclodehydration; the step B (or step B′) subsequent to the step A (or step A′), which is a step of synthesizing an amic acid by adding a diamine other than that employed in the previous step A (or step A′), and then further performing cyclodehydration; and then the step C (or step C′) subsequent to the step B (or step B′), which is a step of reacting a maleic anhydride to synthesize a maleamic acid, and then finally performing cyclodehydration to block molecular chain ends with maleimide groups. The above two production methods mainly differ from each other only in the order in which the different types of diamines are added.

In the above two production methods, the steps can be grouped into two categories which are the synthesis reaction of an amic acid or maleamic acid; and the cyclodehydration reaction. These reactions are described in detail hereunder.

In the step A (or step A′), an amic acid is at first synthesized by reacting a particular tetracarboxylic dianhydride with a particular diamine. This reaction usually proceeds in an organic solvent (e.g. non-polar solvent or high-boiling aprotic polar solvent) and at a temperature of room temperature (25° C.) to 100° C.

Next, the cyclodehydration reaction of the amic acid is performed in a way such that after reacting the amic acid at a temperature of 100 to 160ºC, the cyclodehydration reaction is then caused to proceed while removing from the system a water produced as a by-product due to a condensation reaction. An organic solvent (e.g. non-polar solvent, high-boiling aprotic polar solvent) and/or an acid catalyst may also be added to promote the cyclodehydration reaction.

Examples of the organic solvent include toluene, xylene, anisole, biphenyl, naphthalene, N-methyl-2-pyrrolidone (NMP), N,N-dimethylformamide (DMF), dimethylsulfoxide (DMSO), and dimethylacetamide (DMAC). Any one kind of these organic solvents may be used alone, or two or more kinds of them may be used in combination. Even among them, in terms of solubility, preferred are aromatic solvents such as toluene, xylene, anisole, biphenyl, and naphthalene, of which particularly preferred are toluene, xylene, or anisole.

Examples of the acid catalyst include sulfuric acid, methanesulfonic acid, p-toluenesulfonic acid, and trifluoromethanesulfonic acid. Any one kind of these acid catalysts may be used alone, or two or more kinds of them may be used in combination.

It is preferred that such acid catalyst be used in an amount of 0.1 to 2.0 mol, more preferably 0.2 to 1.0 mol, per 1 mol of the diamine as a raw material.

A molar ratio between the tetracarboxylic dianhydride and the diamine is preferably tetracarboxylic dianhydride/diamine=1.01 to 1.99/1.0, more preferably tetracarboxylic dianhydride/diamine=1.01 to 1.80/1.0, even more preferably tetracarboxylic dianhydride/diamine=1.10 to 1.60/1.0. By combining the tetracarboxylic dianhydride and the diamine at this ratio, there can be synthesized, as a result, a copolymer having an imide group at both ends.

In the step B (or step B′), an amic acid is at first synthesized by reacting the copolymer obtained in the step A (or step A′) with a particular diamine, the copolymer being that having an imide group at both ends. This reaction also usually proceeds in an organic solvent (e.g. non-polar solvent or high-boiling aprotic polar solvent) and at a temperature of room temperature (25° C.) to 100° C.

Likewise, the subsequent cyclodehydration reaction of the amic acid is performed in a way such that after reacting the amic acid at a temperature of 100 to 160° C., the cyclodehydration reaction is then caused to proceed while removing from the system a water produced as a by-product due to a condensation reaction. An organic solvent (e.g. non-polar solvent, high-boiling aprotic polar solvent) and/or an acid catalyst may also be added to promote the cyclodehydration reaction.

Examples of the organic solvent include toluene, xylene, anisole, biphenyl, naphthalene, N-methyl-2-pyrrolidone (NMP), N,N-dimethylformamide (DMF), dimethylsulfoxide (DMSO), and dimethylacetamide (DMAC). Any one kind of these organic solvents may be used alone, or two or more kinds of them may be used in combination. Even among them, in terms of solubility, preferred are aromatic solvents such as toluene, xylene, anisole, biphenyl, and naphthalene, of which particularly preferred are toluene, xylene, or anisole.

Examples of the acid catalyst include sulfuric acid, methanesulfonic acid, p-toluenesulfonic acid, and trifluoromethanesulfonic acid. Any one kind of these acid catalysts may be used alone, or two or more kinds of them may be used in combination.

It is preferred that such acid catalyst be used in an amount of 0.1 to 2.0 mol, more preferably 0.2 to 1.0 mol, per 1 mol of the diamine as a raw material.

A molar ratio between the copolymer having an imide group at both ends and the diamine is preferably 1.0:0.01 to 1.0, more preferably 1.0:0.1 to 1.0.

In the step C (or step C′), a maleamic acid is synthesized by reacting, at a temperature of room temperature (25° C.) to 100° C., a diamine having an amino group at both ends with a maleic anhydride, the diamine being that obtained in the step B (or B′). Finally, cyclodehydration is performed while removing from the system a water produced at 100 to 160° C. as a by-product, thereby blocking the molecular chain ends with maleimide groups, thus obtaining the target bismaleimide compound.

With such production method(s), the bismaleimide compound obtained shall have the structure of a block copolymer, thereby homogenizing and improving the compatibility of the resin synthesized.

A molar ratio between the diamine having an amino group at both ends and the maleic anhydride is preferably 1.0:1.6 to 2.5, more preferably 1.0:1.8 to 2.2.

A solution of the bismaleimide compound obtained by the production method of the present invention can be used to wash a catalyst or the like by a known method (e.g., water, alcohol and the like are added to perform stirring, whereafter the solution stirred is left to stand to separate the organic solvent and aqueous solution.)

The bismaleimide compound obtained by the production method of the present invention can be taken out in the form of a varnish, or purified or isolated into a solid powder via, for example, reprecipitation that is caused by adding a poor solvent. In terms of production cost, it is preferred that the bismaleimide compound obtained by the production method of the present invention be taken out in the form of a varnish. In such case, there can be obtained a resin varnish containing the bismaleimide compound of the present invention and the organic solvent used in the production method thereof. Even as a solvent for the resin varnish of the present invention, there may be listed those that are similar to the organic solvent(s) used in the production method; preferred are aromatic solvents such as toluene, xylene, anisole, biphenyl, and naphthalene, of which particularly preferred are toluene, xylene, or anisole.

### WORKING EXAMPLES

The present invention is described in detail hereunder with reference to working and comparative examples. However, the present invention shall not be limited to the following working examples. In the working and comparative examples, the term “room temperature” means 25° C.

### [Working Example 1] Synthesis of Bismaleimide Compound A-1

Here, 52.27 g (0.150 mol) of 9,9-bis(4-aminophenyl)fluorene (also referred to as FDA hereunder), 104.10 g (0.200 mol) of 4,4′-(4,4′-isopropylidenediphenoxy)diphthalic anhydride, 250 g of toluene, 250 g of N-methyl-2-pyrrolidone, and 9.61 g (0.100 mol) of methanesulfonic acid were added to a 1 L glass four-necked flask equipped with a stirrer, a Dean-Stark tube, a cooling condenser and a thermometer, followed by stirring them at 80° C. for three hours to synthesize an amic acid. Next, the temperature was directly raised to 120° C., and stirring was performed for another eight hours while distilling away a water produced as a by-product, thereby synthesizing a block copolymer.

Later, 53.44 g (0.100 mol) of Priamine-1075 (by CRODA, a dimer diamine expressed by average composition formula H₂N—C₃₆H₇₀—NH₂) was added to the flask containing the block copolymer solution that had been cooled to 80° C., followed by performing stirring at 80° C. for two hours to synthesize an amic acid. Next, the temperature was directly raised to 120° C., and stirring was performed for another eight hours while distilling away a water produced as a by-product, thereby synthesizing a dual-end diamine body.

After cooling the flask containing the dual-end diamine body solution obtained to room temperature, 10.79 g (0.110 mol) of maleic anhydride was added thereto, followed by performing stirring at normal temperature for two hours to synthesize a maleamic acid.

Next, the temperature was directly raised to 120° C., and stirring was performed for another eight hours while distilling away a water produced as a by-product, thereby synthesizing a bismaleimide.

The solution obtained was then washed 10 times with 120 g of a mixed aqueous solution of water and isopropylalcohol to remove impurities such as the catalyst(s), thereby obtaining a varnish solution of the bismaleimide compound. Later, by subjecting the water in the system to azeotropic dehydration together with toluene via reduced-pressure distillation, there was obtained a brown varnish solution with the target bismaleimide being dissolved in toluene (solid content: 50% by mass).

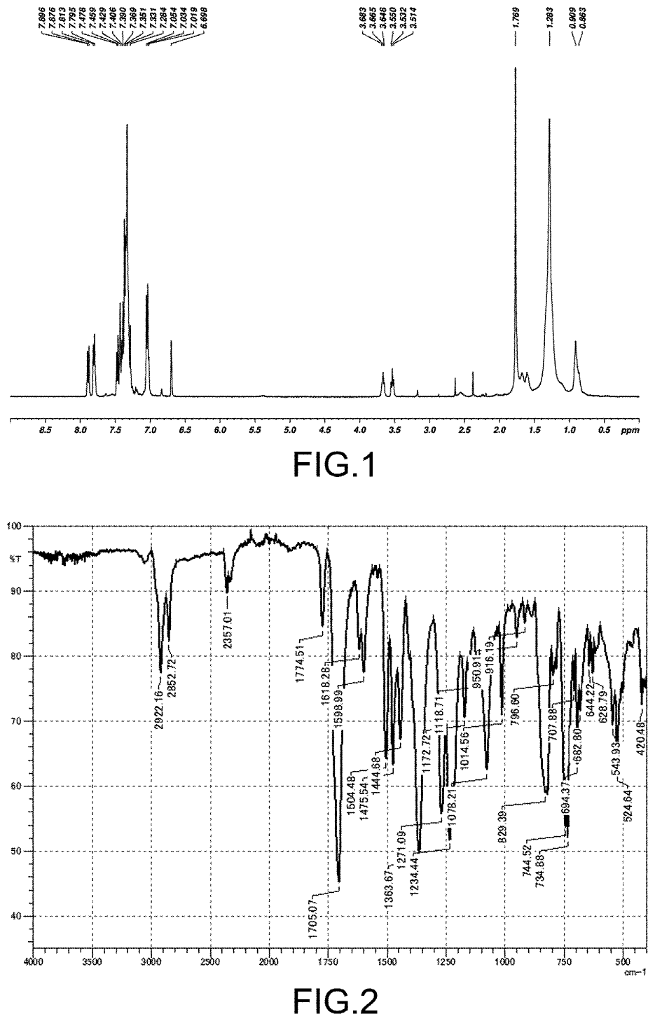

Based on a ¹H-NMR and an IR spectrum of the product obtained, it was confirmed that the product was a bismaleimide compound (number average molecular weight: 6,200) having a structure represented by the following formula (A-1). The ¹H-NMR spectrum of the bismaleimide compound having the structure represented by the formula (A-1) is shown in FIG. 1, and the IR spectrum thereof is shown in FIG. 2.

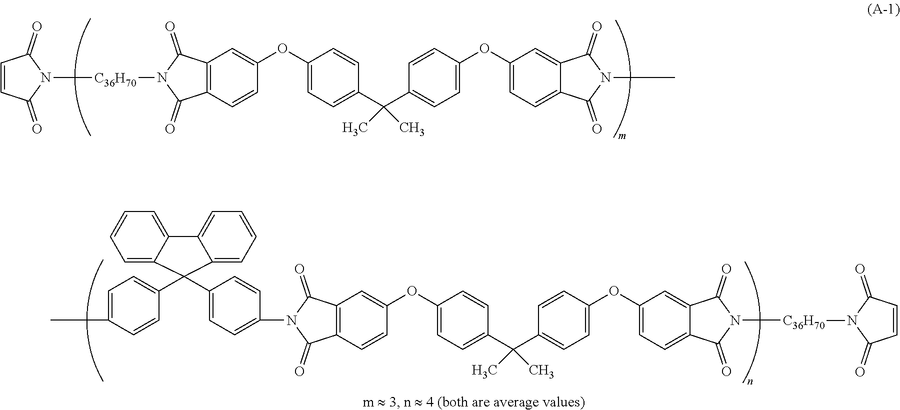

—C₃₆H₇₀— represents a dimer acid frame-derived hydrocarbon group that is derived from dimer diamine (Priamine-1075).

The ¹H-NMR spectrum is shown in FIG. 1. The assignment of each peak is as follows. ¹H-NMR (400 MHZ, CDCl₃) δ=7.90-7.88 (d, 2H), 7.81-7.80 (d, 2H), 7.48-7.46 (d, 2H), 7.43-7.28 (m, 20H), 7.05-7.02 (d, 4H), 6.70 (s, 4H), 3.66 (t, 4H), 3.53 (t, 4H), 1.77 (s, 6H), 1.68-0.86 (m, alkyl group)

The IR spectrum is shown in FIG. 2. Since peaks were observed at 1,705 cm⁻¹ and 1,774 cm⁻¹ indicating the C—O stretching vibration of imide carbonyl, the formation of cyclic imide bonds in the product was confirmed.

### [Working Example 2] Synthesis of Bismaleimide Compound A-2

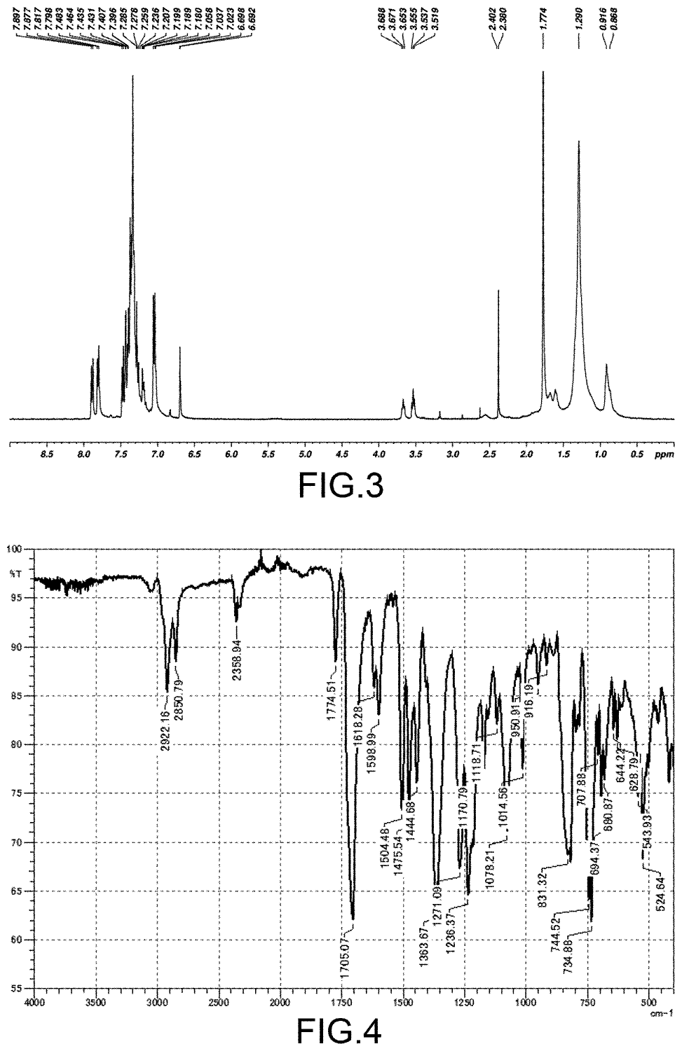

Reactions were performed in a similar manner as the working example 1, except that there were used 56.00 g (0.161 mol) of FDA and 47.71 g (0.089 mol) of Priamine-1075. Based on a ¹H-NMR and an IR spectrum of the product obtained (respectively shown in FIGS. 3 and 4), it was confirmed that the product was a bismaleimide compound (solid content: 50% by weight, number average molecular weight: 6,500) having a structure represented by the following formula (A-2).

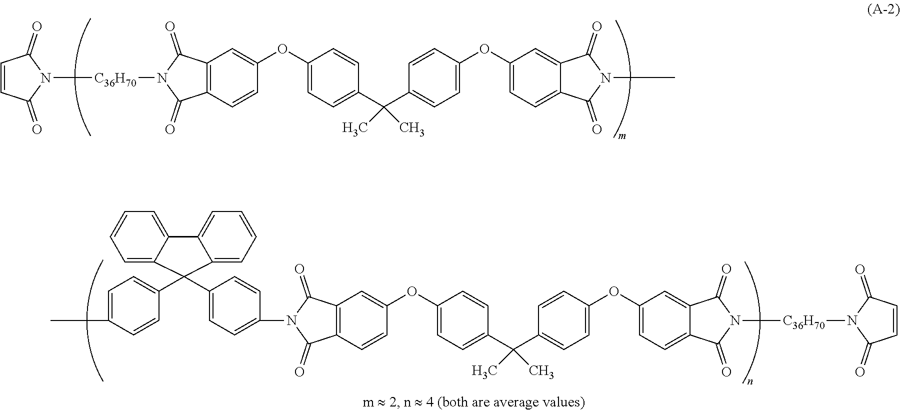

—C₃₆H₇₀— represents a dimer acid frame-derived hydrocarbon group that is derived from dimer diamine (Priamine-1075).

### [Working Example 3] Synthesis of Bismaleimide Compound A-3

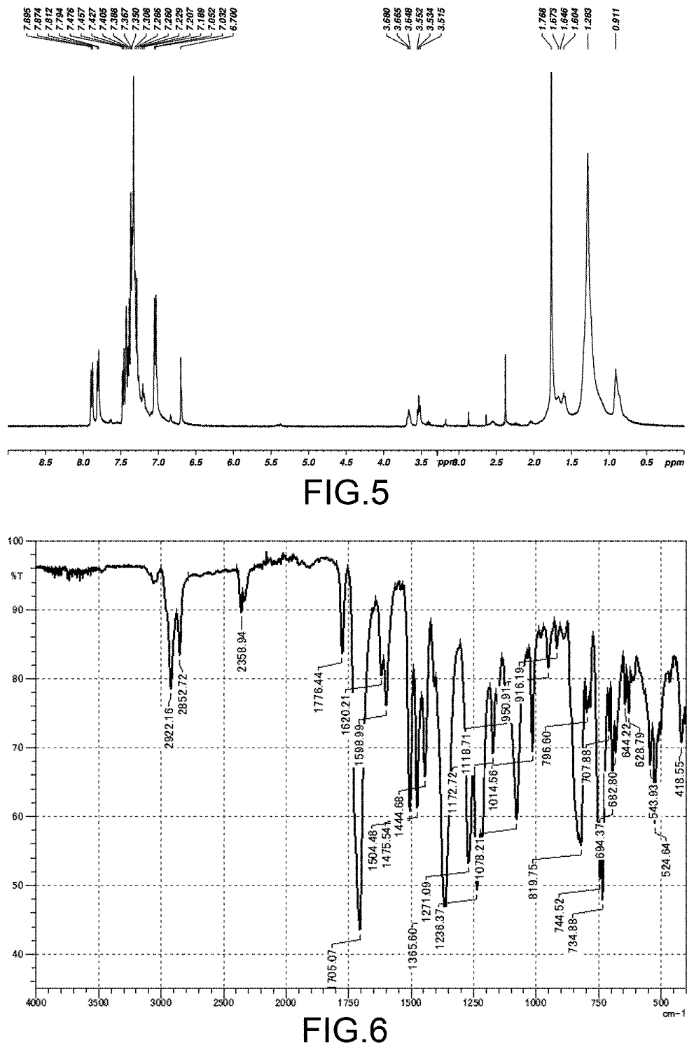

Reactions were performed in a similar manner as the working example 1, except that there were used 58.07 g (0.167 mol) of FDA and 44.53 g (0.083 mol) of Priamine-1075. Based on a ¹H-NMR and an IR spectrum of the product obtained (respectively shown in FIGS. 5 and 6), it was confirmed that the product was a bismaleimide compound (solid content: 50% by weight, number average molecular weight: 6,300) having a structure represented by the following formula (A-3).

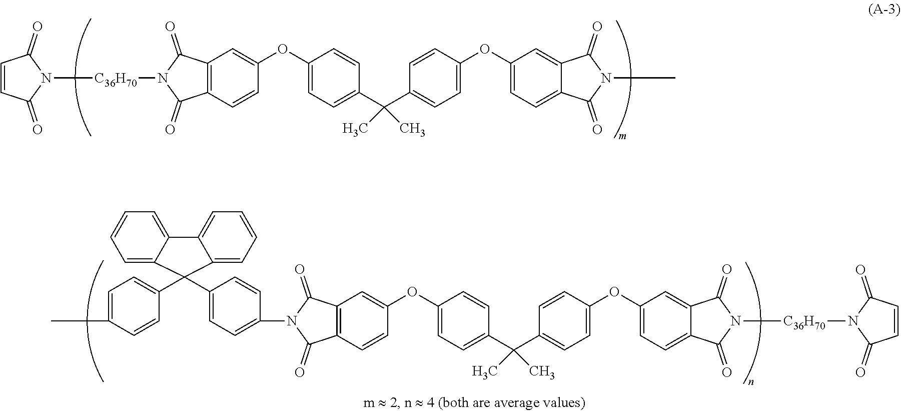

—C₃₆H₇₀— represents a dimer acid frame-derived hydrocarbon group that is derived from dimer diamine (Priamine-1075).

### [Working Example 4] Synthesis of Bismaleimide Compound A-4

Here, 64.07 g (0.167 mol) of 9,9-bis(4-amino-3-fluorophenyl)fluorene, 104.10 g (0.20 mol) of 4,4′-(4,4′-isopropylidenediphenoxy)diphthalic anhydride, 250 g of toluene, 250 g of N-methyl-2-pyrrolidone, and 9.61 g (0.10 mol) of methanesulfonic acid were added to a 1 L glass four-necked flask equipped with a stirrer, a Dean-Stark tube, a cooling condenser and a thermometer, followed by stirring them at 80° C. for three hours to synthesize an amic acid. Next, the temperature was directly raised to 120° C., and stirring was performed for another eight hours while distilling away a water produced as a by-product, thereby synthesizing a block copolymer.

Later, 44.53 g (0.083 mol) of Priamine-1075 (by CRODA, a dimer diamine expressed by average composition formula H₂N—C₃₆H₇₀—NH₂) was added to the flask containing the block copolymer solution that had been cooled to 80° C., followed by performing stirring at 80° C. for two hours to synthesize an amic acid. Next, the temperature was directly raised to 120° C., and stirring was performed for another eight hours while distilling away a water produced as a by-product, thereby synthesizing a dual-end diamine body.

After cooling the flask containing the dual-end diamine body solution obtained to room temperature, 10.79 g (0.110 mol) of maleic anhydride was added thereto, followed by performing stirring at normal temperature for two hours to synthesize a maleamic acid.

Next, the temperature was directly raised to 120° C., and stirring was performed for another eight hours while distilling away a water produced as a by-product, thereby synthesizing a bismaleimide.

The solution obtained was then washed 10 times with 120 g of a mixed aqueous solution of water and isopropylalcohol to remove impurities such as the catalyst(s), thereby obtaining a varnish solution of the bismaleimide compound. Later, by subjecting the water in the system to azeotropic dehydration together with toluene via reduced-pressure distillation, there was obtained a brown varnish solution with the target bismaleimide being dissolved in toluene (solid content: 50% by mass).

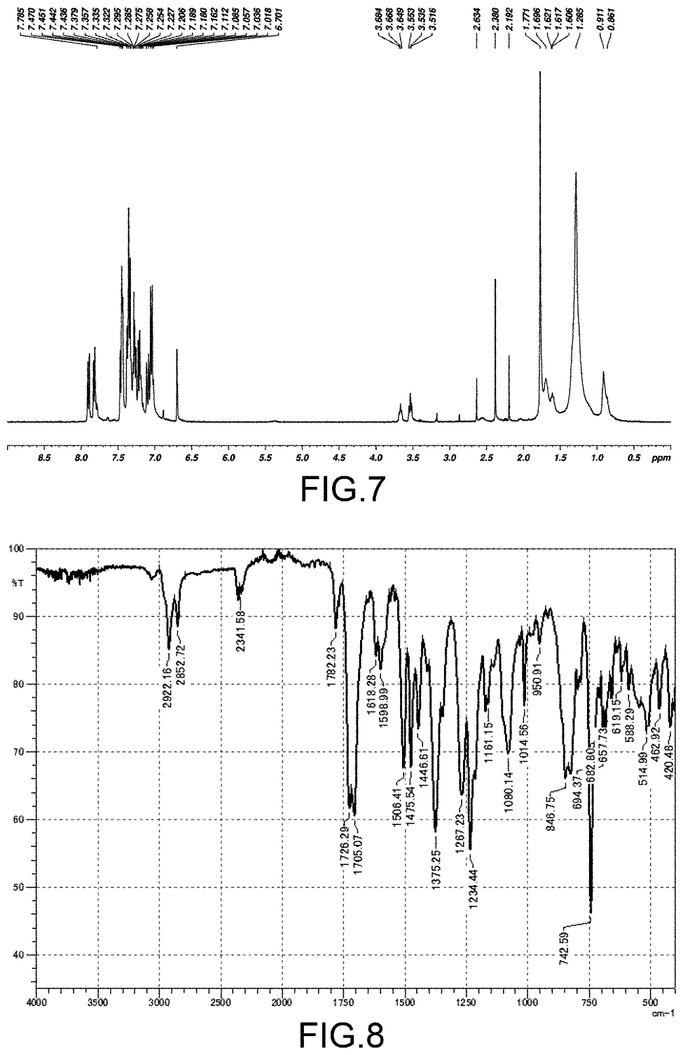

Based on a ¹H-NMR and an IR spectrum of the product obtained (respectively shown in FIGS. 7 and 8), it was confirmed that the product was a bismaleimide compound (solid content: 50% by weight, number average molecular weight: 6,800) having a structure represented by the following formula (A-4).

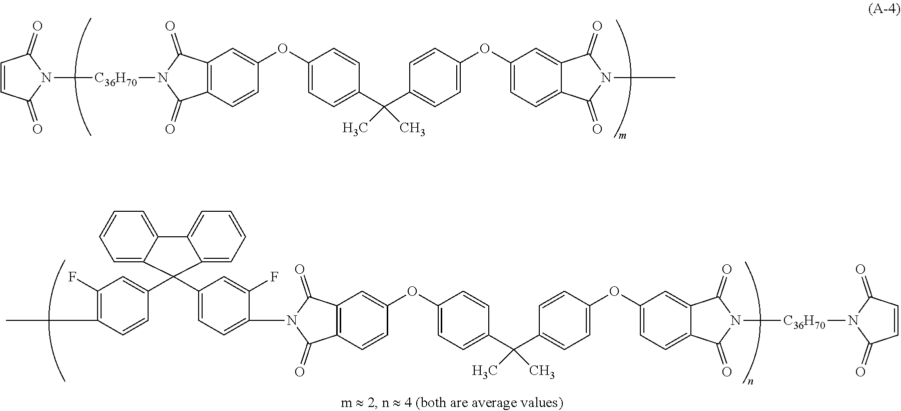

—C₃₆H₇₀— represents a dimer acid frame-derived hydrocarbon group that is derived from dimer diamine (Priamine-1075).

### [Working Example 5] Synthesis of Bismaleimide Compound A-5

Here, 44.40 g (0.167 mol) of 1-(4-aminophenyl)-2,3-dihydro-1,3,3-trimethyl-1H-indene-5-amine, 104.10 g (0.20 mol) of 4,4′-(4,4′-isopropylidenediphenoxy)diphthalic anhydride, 250 g of toluene, 250 g of N-methyl-2-pyrrolidone, and 9.61 g (0.10 mol) of methanesulfonic acid were added to a 1 L glass four-necked flask equipped with a stirrer, a Dean-Stark tube, a cooling condenser and a thermometer, followed by stirring them at 80° C. for three hours to synthesize an amic acid. Next, the temperature was directly raised to 120° C., and stirring was performed for another eight hours while distilling away a water produced as a by-product, thereby synthesizing a block copolymer.

Later, 44.53 g (0.083 mol) of Priamine-1075 (by CRODA, a dimer diamine expressed by average composition formula H₂N—C₃₆H₇₀—NH₂) was added to the flask containing the block copolymer solution that had been cooled to 80° ° C., followed by performing stirring at 80° C. for two hours to synthesize an amic acid. Next, the temperature was directly raised to 120° C., and stirring was performed for another eight hours while distilling away a water produced as a by-product, thereby synthesizing a dual-end diamine body.

After cooling the flask containing the dual-end diamine body solution obtained to room temperature, 10.79 g (0.110 mol) of maleic anhydride was added thereto, followed by performing stirring at normal temperature for two hours to synthesize a maleamic acid.

Next, the temperature was directly raised to 120° C., and stirring was performed for another eight hours while distilling away a water produced as a by-product, thereby synthesizing a bismaleimide.

The solution obtained was then washed 10 times with 120 g of a mixed aqueous solution of water and isopropylalcohol to remove impurities such as the catalyst(s), thereby obtaining a varnish solution of the bismaleimide compound. Later, by subjecting the water in the system to azeotropic dehydration together with toluene via reduced-pressure distillation, there was obtained a brown varnish solution with the target bismaleimide being dissolved in toluene (solid content: 50% by mass).

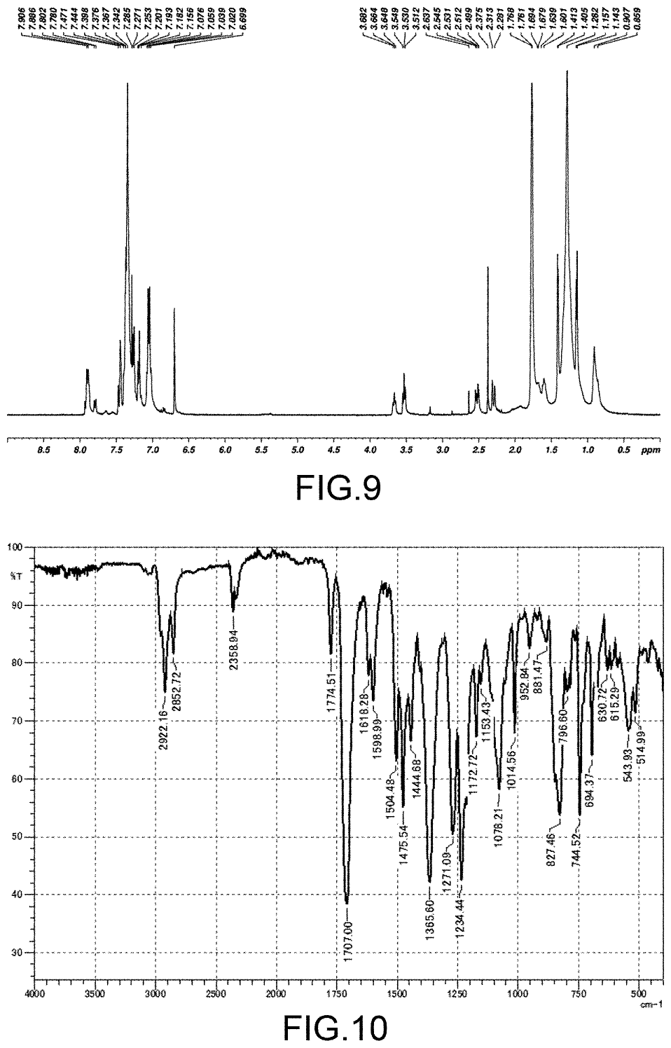

Based on a ¹H-NMR and an IR spectrum of the product obtained (respectively shown in FIGS. 9 and 10), it was confirmed that the product was a bismaleimide compound (solid content: 50% by weight, number average molecular weight: 7,700) having a structure represented by the following formula (A-5).

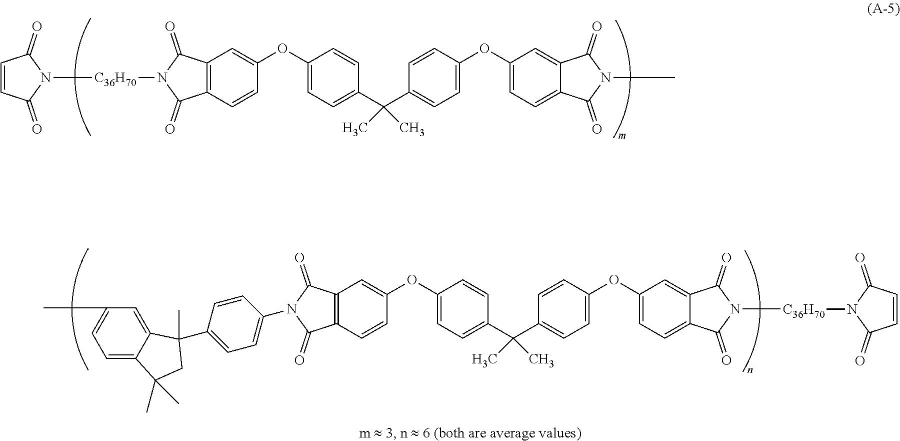

—C₃₆H₇₀— represents a dimer acid frame-derived hydrocarbon group that is derived from dimer diamine (Priamine-1075).

### Bismaleimide Compound

(B-1): Bismaleimide compound represented by the following formula (B-1) (SLK-1400 by Shin-Etsu Chemical Co., Ltd., number average molecular weight: 2,100, for use in comparative example)

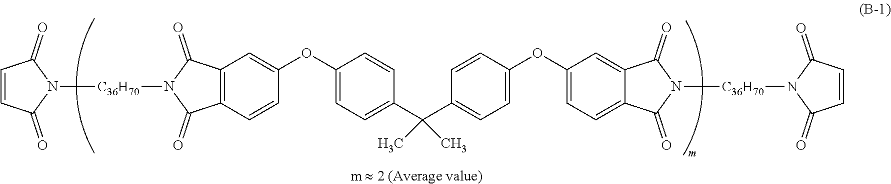

—C₃₆H₇₀— represents a dimer acid frame-derived hydrocarbon group that is derived from dimer diamine (Priamine-1075).

### Bismaleimide Compound

(B-2): Bismaleimide compound represented by the following formula (B-2) (SLK-2700 by Shin-Etsu Chemical Co., Ltd., number average molecular weight: 4,300, for use in comparative example)

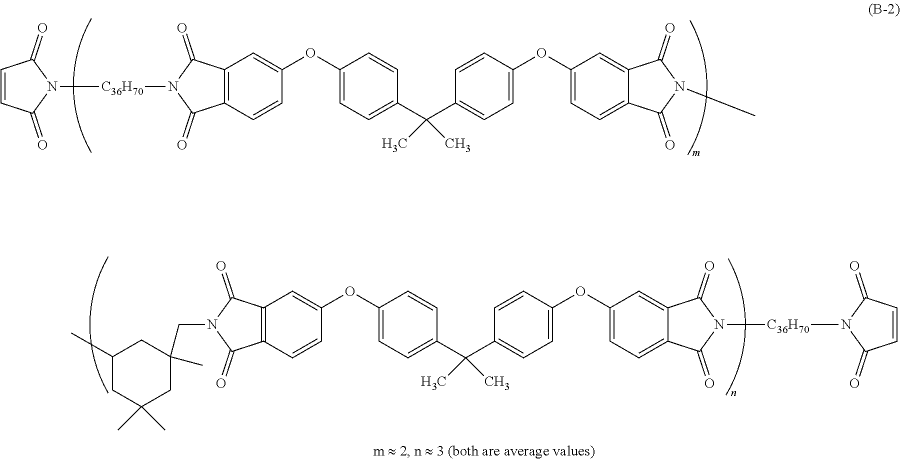

—C₃₆H₇₀— represents a dimer acid frame-derived hydrocarbon group that is derived from dimer diamine (Priamine-1075).

### Bismaleimide Compound

(B-3): Bismaleimide compound represented by the following formula (B-3) (SLK-6200 by Shin-Etsu Chemical Co., Ltd., number average molecular weight: 15,000, for use in comparative example)

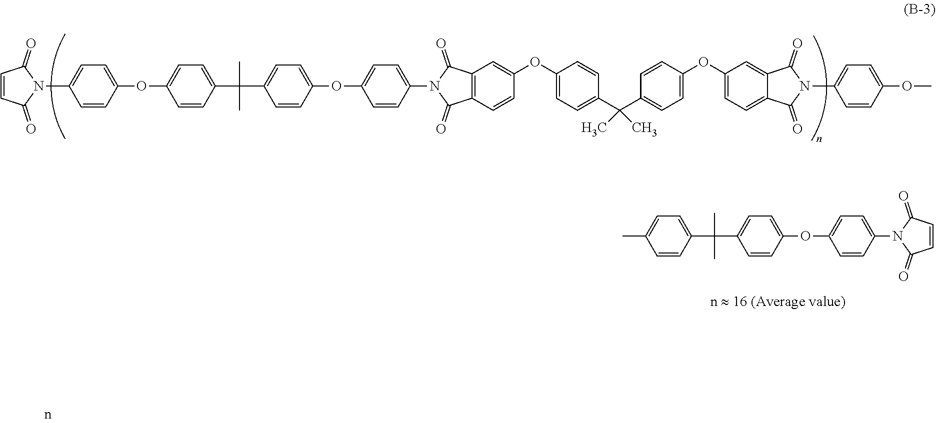

### Bismaleimide Compound

(B-4): Polyphenylmethane maleimide (BMI-2300 by Daiwakasei Industry Co., LTD., for use in comparative example)

### Solvent Solubility of Bismaleimide Compound

Evaluated was a solubility of each of the bismaleimide compounds (A-1) to (A-5) and the bismaleimide compounds (B-1) to (B-4) that were used for comparison in each of anisole, toluene, and xylene. The evaluation results are shown in Table 1.

The method for evaluating the solvent solubility was as follows. Vials prepared by adding each solvent to each bismaleimide compound so that a non-volatile content would be 50% by mass were left to stand at room temperature (25° C.) for 60 days, after which the contents in the vials were visually observed. There, “∘” was given to examples where the compound was uniformly dissolved (no insoluble matter observed), and the content was fluid ; “×” was given to examples where the compound did not dissolve (insoluble matter observed), or the content did not flow.

### Production of Cured Resin Film

The bismaleimide compounds (A-1) to (A-5) obtained in the working examples 1 to 5 and the bismaleimide compounds (B-1) to (B-3) that were used for comparison were each dissolved in toluene or anisole to obtain a toluene varnish solution or anisole varnish solution having a 50% non-volatile content. Next, dicumylperoxide (PERCUMYL D by NOF CORPORATION) was added to this varnish solution by an amount of 0.2 g per 10 g of the solid content, where the solution was thoroughly stirred at room temperature to obtain a varnish-like heat-curable bismaleimide resin composition. A roller coater was then used to apply this varnish solution to a support film composed of a PET film having a thickness of 38 um, followed by drying the same at 120° C. for 10 min to remove the solvent, thereby forming an 80 um-thick uncured resin film on the support film. Further, by performing curing at 180° C. under a nitrogen atmosphere for two hours, there was obtained a cured resin film.

Dicumylperoxide (PERCUMYL D by NOF CORPORATION) was added to and mixed with the bismaleimide compound (B-4) used for comparison, by an amount of 0.2 g per 10 g of the resin content, thereby obtaining a resin composition. There was prepared a frame having a size of 60 mm×60 mm and a thickness of 200 μm. The resin composition was then sandwiched by two pieces of mold release-treated PET films (E7006 by TOYOBO Co.,LTD.) each having a thickness of 75 μm, and a vacuum press machine (Nikko-Materials Co., Ltd.) was used to perform molding at 180° C. for 5 min to obtain a cured product (molded film). This molded film was then subjected to post curing at 180° C. under a nitrogen atmosphere for two hours to obtain a cured resin film.

### Flexibility of Cured Resin Film

The cured resin film was bended at 180° five times, where “∘” was given to examples exhibiting no flaws in the film such as cracks, and “×” was given to examples exhibiting flaws in the film such as cracks. The results are shown in Table 1.

### Relative Permittivity, Dielectric Tangent

Connected to a network analyzer (E5063-2D5 by Keysight Technologies) and a stripline (by KEYCOM Corp.), the cured resin film was dried at 120° C. for an hour and then stored in a room of 25° C. and a humidity of 50% for 24 hours before having its relative permittivity and dielectric tangent at frequencies of 10 GHz and 40 GHz measured. The results are shown in Table 1.

### Moisture Resistance Test

In order to check a moisture resistance, the cured resin film was left under a temperature of 85° C. and a humidity of 85% for 1,000 hours before having its relative permittivity and dielectric tangent at the frequency of 10 GHz measured in a similar manner as above. The results are shown in Table 1.

### Heat Resistance Test

In order to check a high-temperature heat resistance, the cured resin film was left at 150° C. under air atmosphere for 1,000 hours before having its relative permittivity and dielectric tangent at the frequency of 10 GHz measured in a similar manner as above. The results are shown in Table 1.

### Glass-Transition Temperature

A storage elastic modulus (MPa) of the cured resin film was measured by DMA Q800 (by TA Instruments, Inc.) over a range of −20 to 300° C.; defined as a glass-transition temperature (Tg) was a peak top temperature obtained from a graph in which Tan δ values derived from the values of the storage elastic modulus obtained and a loss elastic modulus were plotted. The measurement was conducted under conditions of: sample having a size of 40 mm×5 mm and a thickness of 80 μm; rate of temperature increase 5° C./min; frequency 10 Hz; tensile mode; amplitude 15 μm. The results are shown Table 1.

### Thermal Expansion Coefficient

The thermal expansion coefficient (CTE) of the cured resin film was measured by TMA Q400 (by TA Instruments, Inc.) over a range of −50 to 300° C., where the thermal expansion coefficient was calculated in a range of 0 to 40° C. The measurement was conducted under conditions of: sample having a size of 30 mm×3 mm and a thickness of 80 μm; rate of temperature increase 5° C./min; test load 0.075 N. The results are shown Table 1.

TABLE 1

| | | | Working | Working | Working | Working | Working |
|---|---|---|---|---|---|---|---|
| | | | example 1 | example 2 | example 3 | example 4 | example 5 |
| | Bismaleimide compound | | A-1 | A-2 | A-3 | A-4 | A-5 |
| Evaluation | Solubility | Anisole | ◯ | ◯ | ◯ | ◯ | ◯ |
| results | | Toluene | ◯ | ◯ | ◯ | ◯ | ◯ |
| | | Xylene | ◯ | ◯ | ◯ | ◯ | ◯ |

| | | | | | |
|---|---|---|---|---|---|
| Flexibility of cured resin film | ◯ | ◯ | ◯ | ◯ | ◯ |
| Relative permittivity (10 GHz) | 2.7 | 2.7 | 2.8 | 2.8 | 2.7 |
| Dielectric tangent (10 GHz) | 0.0019 | 0.0022 | 0.0024 | 0.0025 | 0.0022 |
| Relative permittivity (40 GHz) | 2.7 | 2.7 | 2.7 | 2.8 | 2.7 |
| Dielectric tangent (40 GHz) | 0.0025 | 0.0028 | 0.0031 | 0.0033 | 0.0029 |
| Relative permittivity after | 2.7 | 2.7 | 2.8 | 2.8 | 2.7 |
| moisture resistance test (10 GHz) | | | | | |
| Dielectric tangent after moisture | 0.0025 | 0.0027 | 0.0030 | 0.0030 | 0.0026 |
| resistance test (10 GHz) | | | | | |
| Relative permittivity after heat | 2.7 | 2.7 | 2.8 | 2.8 | 2.7 |
| resistance test (10 GHz) | | | | | |
| Dielectric tangent after heat | 0.0035 | 0.0034 | 0.0032 | 0.0033 | 0.0035 |
| resistance test (10 GHz) | | | | | |
| Glass-transition temperature | 190 | 202 | 213 | 214 | 183 |
| (DMA)[° C.] | | | | | |
| Thermal expansion coefficient | 74 | 70 | 67 | 69 | 74 |
| [ppm/° C.] | | | | | |

| | | | Comparative | Comparative | Comparative | Comparative |
|---|---|---|---|---|---|---|
| | | | example 1 | example 2 | example 3 | example 4 |
| | Bismaleimide compound | | B-1 | B-2 | B-3 | B-4 |
| Evaluation | Solubility | Anisole | ◯ | ◯ | ◯ | X |
| results | | Toluene | ◯ | ◯ | X | X |
| | | Xylene | ◯ | ◯ | X | X |

| | | | | |
|---|---|---|---|---|
| Flexibility of cured resin film | ◯ | ◯ | ◯ | X |
| Relative permittivity (10 GHz) | 2.5 | 2.6 | 2.9 | 3.3 |
| Dielectric tangent (10 GHz) | 0.0019 | 0.0021 | 0.0050 | 0.0220 |
| Relative permittivity (40 GHz) | 2.5 | 2.6 | 2.9 | 3.5 |
| Dielectric tangent (40 GHz) | 0.0019 | 0.0028 | 0.0062 | 0.0550 |
| Relative permittivity after | 2.5 | 2.6 | 3.0 | 3.5 |
| moisture resistance test (10 GHz) | | | | |
| Dielectric tangent after moisture | 0.0024 | 0.0024 | 0.0065 | 0.0345 |
| resistance test (10 GHz) | | | | |
| Relative permittivity after heat | 2.6 | 2.7 | 2.9 | 3.5 |
| resistance test (10 GHz) | | | | |
| Dielectric tangent after heat | 0.0070 | 0.0061 | 0.0055 | 0.0270 |
| resistance test (10 GHz) | | | | |
| Glass-transition temperature | 40 | 165 | 200 | 250< |
| (DMA)[° C.] | | | | |
| Thermal expansion coefficient | 110 | 75 | 50 | 38 |
| [ppm/° C.] | | | | |

The above results indicate that the bismaleimide compound of the present invention has a favorable solubility in various solvents, particularly in a non-polar solvent such as toluene; and that a cured resin film containing such bismaleimide compound has a favorable flexibility, a low relative permittivity and dielectric tangent, an excellent moisture resistance and heat resistance, a high glass-transition temperature, and a low thermal expansion coefficient.

Especially, as compared to the comparative example 2 where there was employed a compound having the same resin frame as the bismaleimide compound of the present invention except that the aromatic ring-containing divalent group in the bismaleimide compound of the present invention was replaced by an alicyclic structure-containing divalent group, the bismaleimide compound of the present invention resulted in a high glass-transition temperature, and small changes in dielectric properties and an excellent heat resistance even under a high-temperature environment.

### INDUSTRIAL APPLICABILITY

If using the bismaleimide compound of the present invention, there can be provided a bismaleimide resin composition capable of being turned into a cured product having a low thermal expansion coefficient, a high glass-transition temperature (Tg), and an excellent moisture resistance and heat resistance so much so that the cured product shall have a low relative permittivity and dielectric tangent and thus a high reliability even under a high-temperature environment. Further, since the bismaleimide compound of the present invention is superior in solvent solubility, when added to a bismaleimide resin composition, the compound can be easily used in combination with other resins, and better performances can thereby be easily elicited by supplementing each other's performances. Specifically, the bismaleimide compound of the present invention is suitable for use in, for example, a multilayered printed wiring board installed in a high-frequency band electronic device requiring an insulation material with superior dielectric properties.

What is claimed is:

1. A bismaleimide compound represented by the following formula (1): 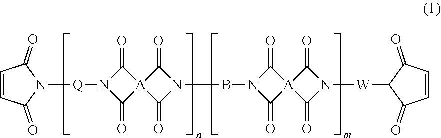
wherein A independently represents a cyclic structure-containing tetravalent organic group; B independently represents a dimer acid frame-derived divalent hydrocarbon group; Q independently represents an aromatic ring-containing divalent group; W is B or Q; n is 1 to 100; m is 1 to 100; no restrictions are imposed on an order of each repeating unit identified by n and m, and a bonding pattern thereof may be alternate, block or random, and
wherein Q has a fluorene frame or indene frame expressed by any of the following formula (2-1) or (2-2): 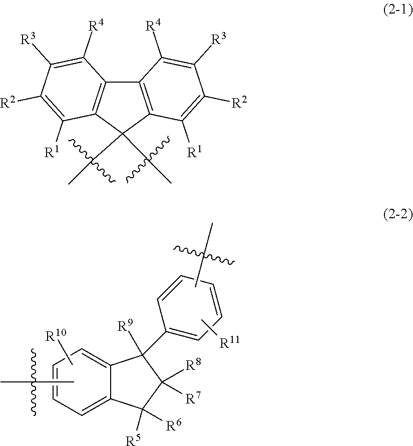
wherein in the formula (2-1), each of R¹, R², R³ and R⁴ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group, and
wherein in the formula (2-2), each of R⁵, R⁶, R⁷, R⁸, R⁹, R¹⁰ and R¹¹ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group.

2. The bismaleimide compound according to claim 1, wherein A in the formula (1) is a tetravalent organic group represented by the following structural formula (5): 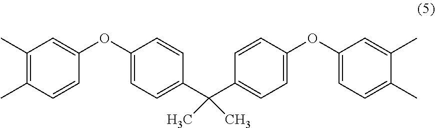
wherein bonds that are yet unbonded to substituent groups are to be bonded to carbonyl carbons forming cyclic imide structures in the formula (1).

3. The bismaleimide compound according to claim 1, wherein the bismaleimide compound represented by the formula (1) has a number average molecular weight of 3,000 to 50,000.

4. The bismaleimide compound according to claim 1, wherein in the bismaleimide compound represented by the formula (1), the bonding pattern of each repeating unit identified by n and m is block.

5. A resin varnish containing the bismaleimide compound according to claim 1 and a solvent.

6. The resin varnish according to claim 5, wherein the solvent is an aromatic solvent.

7. The resin varnish according to claim 6, wherein the aromatic solvent is toluene, xylene, or anisole.

8. A method for producing the bismaleimide compound according to claim 1, comprising:
a step A of synthesizing an amic acid with an acid anhydride represented by the following formula (6) and an aromatic ring-containing diamine represented by the following formula (7), and then performing cyclodehydration;
a step B subsequent to the step A, which is a step of synthesizing an amic acid with a reactant obtained in the step A and a dimer acid frame-derived diamine represented by the following formula (8), and then performing cyclodehydration; and
a step C subsequent to the step B, which is a step of synthesizing a maleamic acid with a reactant obtained in the step B and a maleic anhydride, and then performing cyclodehydration to block molecular chain ends, 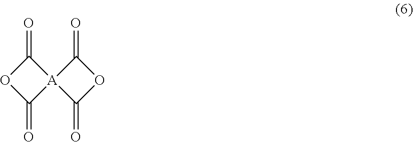
wherein A represents a cyclic structure-containing tetravalent organic group;
H₂N-Q-NH₂ (7)
wherein Q represents an aromatic ring-containing divalent group that has a fluorene frame or indene frame expressed by any of the following formula (2-1) or (2-2), 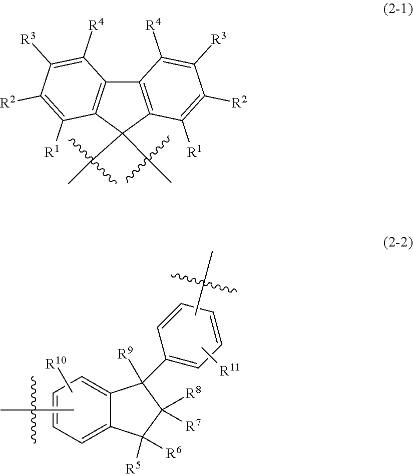
wherein in the formula (2-1), each of R¹, R², R³ and R⁴ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group, and
wherein in the formula (2-2), each of R⁵, R⁶, R⁷, R⁸, R⁹, R¹⁰ and R¹¹ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group;
H₂N—B—NH₂ (8)
wherein B represents a dimer acid frame-derived divalent hydrocarbon group.

9. A method for producing the bismaleimide compound according to claim 1, comprising:
a step A′ of synthesizing an amic acid with an acid anhydride represented by the following formula (6) and a diamine represented by the following formula (8), and then performing cyclodehydration;
a step B′ subsequent to the step A′, which is a step of synthesizing an amic acid with a reactant obtained in the step A′ and an aromatic ring-containing diamine represented by the following formula (7), and then performing cyclodehydration; and
a step C′ subsequent to the step B′, which is a step of synthesizing a maleamic acid with a reactant obtained in the step B′ and a maleic anhydride, and then performing cyclodehydration to block molecular chain ends, 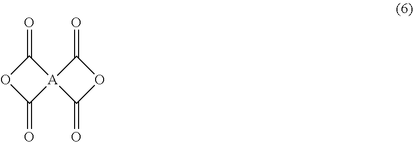
wherein A represents a cyclic structure-containing tetravalent organic group;
H₂N-Q-NH₂ (7)
wherein Q represents an aromatic ring-containing divalent group that has a fluorene frame or indene frame expressed by any of the following formula (2-1) or (2-2), 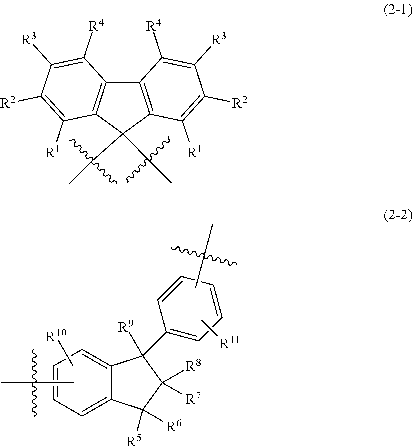
wherein in the formula (2-1), each of R¹, R², R³ and R⁴ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group, and
wherein in the formula (2-2), each of R⁵, R⁶, R⁷, R⁸, R⁹, R¹⁰ and R¹¹ independently represents a hydrogen atom, an alkyl group having 1 to 5 carbon atoms, a (hetero)aryl group having 4 to 10 carbon atoms, a hydroxyl group, an alkoxy group, a halogeno group, a trifluoromethyl group, an amino group, or a sulfenyl group;
H₂N—B—NH₂ (8)
wherein B represents a dimer acid frame-derived divalent hydrocarbon group.
